(19) United States
(12) Patent Application Publication
(10) Pub. No.: US 2010/0241013 A1
(43) Pub. Date: Sep. 23, 2010

(54) Direct Measurements of Arterial Pressure Decoupling

(75) Inventor: Feras Hatib, Irvine, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/718,539

(22) Filed: Mar. 5, 2010

Related U.S. Application Data
(60) Provisional application No. 61/161,120, filed on Mar. 18, 2009.

(51) Int. Cl. A61B 5/021

(52) U.S. Cl. 600/485

(57) ABSTRACT

Methods for monitoring central-to-peripheral arterial pressure decoupling, i.e., hyperdynamic or vasodilation conditions are described. These methods involve the comparison of parameters such as impedance, compliance, and pressure that can be determined from flow and pressure measurements at central aortic and peripheral arterial locations. The relationship between the parameters at the central aortic and peripheral arterial locations provides an indication of central-to-peripheral arterial pressure decoupling. These methods can be alert a user that a subject is experiencing central-to-peripheral arterial pressure decoupling, which can enable a clinician to appropriately provide treatment to the subject.

### CLAIM OF PRIORITY UNDER 35 U.S.C. §119

The application claims the benefit of U.S. Provisional Application No. 61/161,120 filed Mar. 18, 2009, entitled “Direct Measurements of Arterial Pressure Decoupling” and assigned to the assignee hereof and hereby incorporated by reference in its entirety.

### BACKGROUND

Indicators such as stroke volume (SV), cardiac output (CO), end-diastolic volume, ejection fraction, stroke volume variation (SVV), pulse pressure variation (PPV), and systolic pressure variations (SPV), among others, are important not only for diagnosis of disease, but also for “real-time,” i.e., continual, monitoring of clinically significant changes in a subject. For example, health care providers are interested in changes in preload dependence, fluid responsiveness, or volume responsiveness as well as, for example, central-to-peripheral decoupling in both human and animal subjects. Few hospitals are therefore without some form of equipment to monitor one or more cardiac indicators in an effort to provide a warning that one or more of the indicated changes are occurring in a subject. Many techniques, including invasive techniques, non-invasive techniques, and combinations thereof, are in use and even more have been proposed in the literature.

### SUMMARY

Methods for monitoring central-to-peripheral arterial pressure decoupling in a subject are described. The methods involve the comparison of parameters such as vascular impedance, arterial compliance, and central aortic and peripheral arterial pressures that can be determined from flow and pressure measurements at central aortic and peripheral arterial locations. The relationship between these parameters provides an indication of central-to-peripheral arterial pressure decoupling.

The methods for monitoring central-to-peripheral arterial pressure decoupling in a subject using vascular impedance involve measuring central aortic pressure, central aortic flow, and peripheral arterial pressure in the subject. Then calculating a central systemic vascular impedance from the central aortic pressure and the central aortic flow, and a peripheral systemic vascular impedance from the peripheral arterial pressure and the central aortic flow. The central systemic vascular impendence is compared to the peripheral systemic vascular impedance, and central-to-peripheral arterial pressure decoupling is indicated if the subject's peripheral systemic vascular impedance is greater than the subject's central systemic vascular impedance.

The methods for monitoring central-to-peripheral arterial pressure decoupling in a subject using arterial compliance involve measuring central aortic pressure, central aortic flow, and peripheral arterial pressure in the subject. Then calculating a central systemic arterial compliance from the central aortic pressure and the central aortic flow, and a peripheral systemic arterial compliance from the peripheral arterial pressure and the central aortic flow. The central systemic arterial compliance is compared to the peripheral systemic arterial compliance, and central-to-peripheral arterial pressure decoupling is indicated if the subject's peripheral systemic arterial compliance is greater than the subject's central systemic arterial compliance.

The methods for monitoring central-to-peripheral arterial pressure decoupling in a subject using central aortic pressure and peripheral arterial pressure involve measuring the subject's central aortic pressure and the subject's peripheral arterial pressure. The subject's central aortic pressure is compared to the subject's peripheral arterial pressure, and central-to-peripheral arterial pressure decoupling is indicated if the subject's central aortic pressure is greater than the subject's peripheral arterial pressure.

### DESCRIPTION OF DRAWINGS

FIG. 1 shows simultaneously recorded pressure waveforms in the ascending aorta (Aortic), femoral artery (Femoral), and radial artery (Radial) in a porcine animal model during normal hemodynamic conditions.

FIG. 2 shows simultaneously recorded pressure waveforms in the ascending aorta (Aortic), femoral artery (Femoral), and radial artery (Radial) in a porcine animal model during Endotoxin shock (septic shock) resuscitated with large amounts of fluids and vasopressors.

FIG. 3 shows a flow chart illustrating an example of logic for monitoring central-to-peripheral arterial pressure decoupling in a subject using central systemic vascular impendence and peripheral systemic vascular impedance.

FIG. 4 shows a flow chart illustrating an example of logic for monitoring central-to-peripheral arterial pressure decoupling in a subject using central systemic arterial compliance and peripheral systemic arterial compliance.

FIG. 5 shows a two-element Compliance-Resistance model of the arterial system.

FIG. 6 shows a flow chart illustrating an example of logic for monitoring central-to-peripheral arterial pressure decoupling in a subject using central aortic pressure and peripheral arterial pressure.

FIGS. 7A-C show plots of impedance (7A), resistance (7B), and compliance (7C) measurements for a subject in a normal hemodynamic condition then in a peripherally decoupled condition.

FIG. 8 is a block diagram showing the main components of a system to implement the methods described herein.

### DETAILED DESCRIPTION

Methods for monitoring central-to-peripheral arterial pressure decoupling, i.e., hyperdynamic hemodynamic conditions are described. These methods involve the comparison of parameters such as impedance, compliance, and pressure that can be determined from flow and pressure measurements at central aortic and peripheral arterial locations. The relationship between the parameters at the central aortic and peripheral arterial locations provides an indication of central-to-peripheral arterial pressure decoupling. Specifically, when peripheral impedance or pressure values fall to levels below the analogous central impedance or pressure parameter values (or vice versa for compliance), central-to-peripheral arterial pressure decoupling is indicated. These methods can alert a user that a subject is experiencing central-to-peripheral arterial pressure decoupling, which can enable a clinician to appropriately provide treatment to the subject.

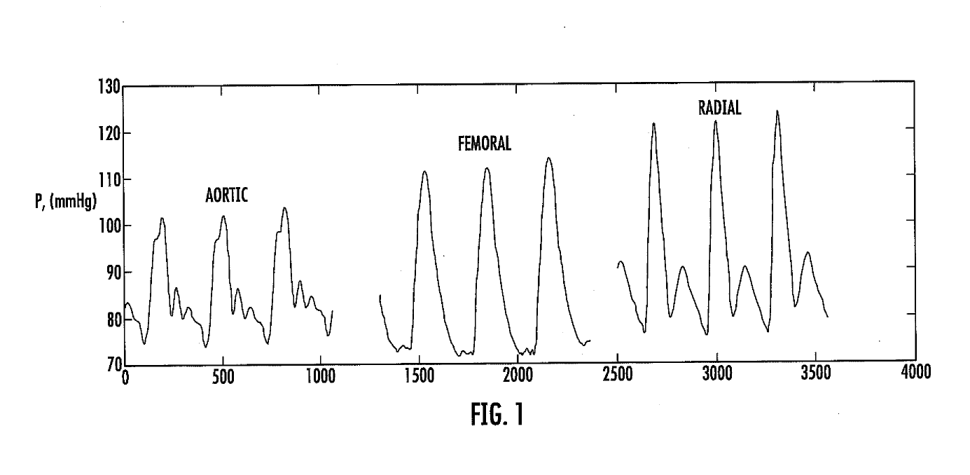

As used herein, the phrases hyperdynamic and vasodilation mean a condition in which the arterial peripheral pressure and flow are decoupled from the central aortic pressure and flow, and the term peripheral arteries is intended to mean arteries located away from the heart, e.g., radial, femoral, or brachial arteries. Decoupled arterial pressure means that the normal relationship between the arterial peripheral pressure and the central aortic pressure is not valid and the arterial and peripheral arterial pressure can not be used to determine the central arterial pressure. This also includes conditions in which the peripheral arterial pressure is not proportional or is not a function of the central aortic pressure. Under normal hemodynamic conditions, blood pressure increases the further away from the heart the measurement is taken. Such a pressure increase is shown in FIG. 1, i.e., the amplitude of a pressure wave measured at radial arteries is greater than the pressure measured at the femoral artery, which in turn is greater than the aortic pressure. These differences in pressure are related to wave reflection, i.e., pressure is amplified toward the periphery.

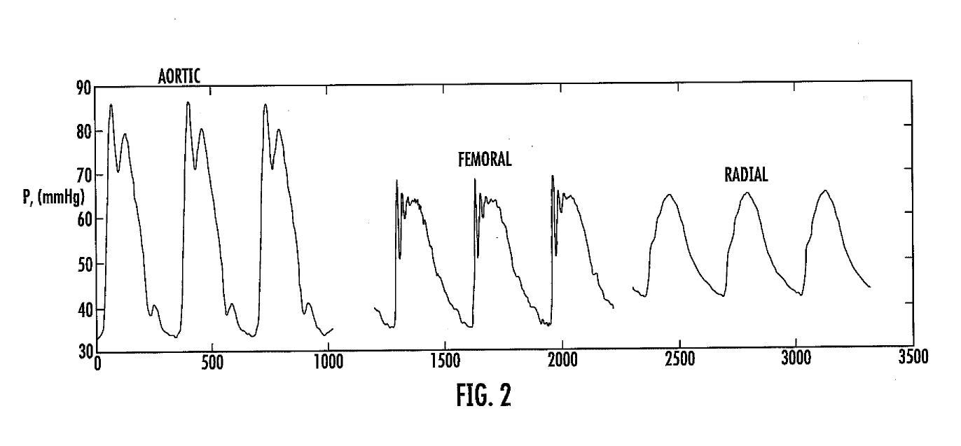

This normal hemodynamic relationship of pressures, i.e., an increase in pressure away from the heart, is often relied upon in medical diagnosis. However, under hyperdynamic/vasodilation conditions, this relationship can become inverted with the arterial pressure becoming lower than the central aortic pressure. This reversal has been attributed, for example, to arterial tone in the peripheral vessels, which is suggested to impact the wave reflections in the arterial system. Such a hyperdynamic condition is shown in FIG. 2, i.e., the amplitude of a pressure wave measured at radial arteries is lower than the pressure measured as the femoral artery, which in turn is lower than the aortic pressure. Drugs that dilate small peripheral arteries (e.g., nitrates, ACE inhibitors, and calcium inhibitors) are thought to contribute to this effect. These types of severe vasodilatory conditions are also often observed in situations right after cardiopulmonary bypass (coronary bypass), in which the radial arterial pressure underestimates the pressure in the aorta. Substantial central to peripheral pressure differences, where the peripheral arterial pressure underestimates the central aortic pressure, are usually observed in patients with severe sepsis who are treated with large amount of fluids and high-dose vasopressors, leading to severe vasodilation. Very similar conditions are also observed in patients with end stage liver disease. As will be well appreciated by those of skill in the art, certain treatments for subjects in normal hemodynamic conditions will be approached differently than for subjects in hyperdynamic conditions. Thus, the presently disclosed methods detect central-to-peripheral arterial pressure decoupling, i.e., vasodilation in a subject, if present.

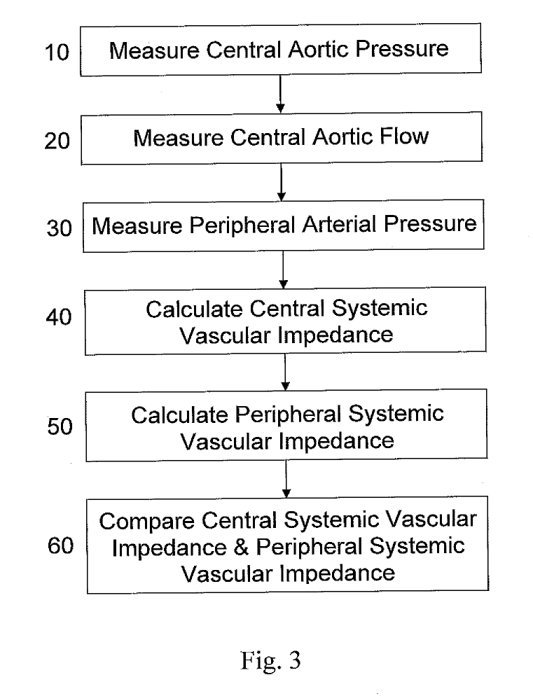

A first method for monitoring central-to-peripheral arterial pressure decoupling in a subject is shown as a flow chart in FIG. 3 and involves measuring the central aortic pressure (10), the central aortic flow (20), and the peripheral arterial pressure (30) of a subject. Next the central systemic vascular impedance is calculated (40) by dividing the central aortic pressure by the central aortic flow and the peripheral systemic vascular impedance is calculated (50) by dividing the peripheral arterial pressure by the central aortic flow. Then the central systemic vascular impendence is compared to the peripheral systemic vascular impedance (60). If the subject's peripheral systemic vascular impedance is greater than the subject's central systemic vascular impedance then central-to-peripheral arterial pressure decoupling is indicated. The degree of central-to-peripheral arterial pressure decoupling in subjects in which central-to-peripheral arterial pressure decoupling is indicated can be determined by subtracting the peripheral systemic vascular impedance from the central systemic vascular impendence. Whether a subject is experiencing central-to-peripheral arterial pressure decoupling and/or the degree of central-to-peripheral arterial pressure decoupling can be continuously monitored by continuously monitoring the central systemic vascular impedance and peripheral systemic vascular impedance.

Central systemic vascular impedance can be calculated by dividing the central aortic pressure by the central aortic flow as follows:

$${Z}_{a} ( jω ) = \frac{{P}_{a} ( jω )}{{Q}_{a} ( jω )}$$

where Zₐ is systemic vascular impedance (the subscript a indicates that the measurement is performed at the level of the aorta), Pₐ is the power spectrum of aortic pressure, Qₐ is the power spectrum of aortic flow, j is the imaginary unit, indicating a complex function, and the frequency, ω, is 2πf. As will be clear to those of skill in the art, all the mathematical operations described here are performed in the frequency domain. Any number of harmonics of the pressure signal or the flow signal can be used, e.g., the first 10, or the first 20 harmonics of the pressure and flow signals. The power spectrum of the pressure and flow signals could be calculated, e.g., with a Fast Fourier Transform (FFT). Other methods to calculate the power spectrum of a signal are known to those of skill in the art. Similarly, peripheral arterial impedance can be calculated by dividing the peripheral arterial pressure by the central aortic flow as follows:

$${Z}_{p} ( jω ) = \frac{{P}_{p} ( jω )}{{Q}_{a} ( jω )}$$

where Zₚ is peripheral systemic vascular impedance (the subscript p indicating the measurement is performed in a peripheral vessel), Pₚ is the power spectrum of the peripheral arterial pressure, Qₐ is the power spectrum of the peripheral arterial flow, j is the imaginary unit, indicating a complex function, and the frequency, ω, is 2πf. Whether a subject's peripheral systemic vascular impedance is greater than the subject's central systemic vascular impedance (i.e., whether central-to-peripheral arterial pressure decoupling is indicated (PDT)) can be expressed mathematically as follows:

PDI=Zₐ(jω)>Zₚ(jω)

The degree of central-to-peripheral arterial pressure decoupling in subjects in which central-to-peripheral arterial pressure decoupling is indicated can be expressed mathematically as follows:

$$PD = \{ \begin{matrix}{Z}_{a} ( jω ) - {Z}_{p} ( jω ) & for & {Z}_{a} ( jω ) > {Z}_{p} ( jω ) \\ 0 & for & {Z}_{a} ( jω ) \leq {Z}_{p} ( jω )\end{matrix} \}$$

In this relationship, the degree of the peripheral decoupling will be shown as 0 when the central systemic vascular impedance is lower than the peripheral systemic vascular impedance (i.e. no peripheral decoupling is indicated) and the degree of peripheral decoupling will be a equal to the difference between the central systemic vascular impedance and the peripheral systemic vascular impedance when the central systemic vascular impedance is greater than the peripheral systemic vascular impedance (i.e. peripheral decoupling is indicated).

Further, the difference between the central and peripheral systemic vascular impedances can be measured continuously, which will indicate the degree of the difference between the central systemic vascular impedance and the peripheral systemic vascular impedance.

PD=Zₐ(jω)−Zₚ(jω)

When using this relationship, if PD is greater than zero, peripheral pressure decoupling is indicated, and the greater the value of PD, the greater the peripheral decoupling. When PD is less than zero, normal conditions are indicated. If PD is less than zero by more than 25%, peripheral vasoconstriction will be indicated.

The degree of peripheral decoupling could be given in % as follows:

$$PD = \{ \begin{matrix}\frac{{Z}_{a} ( jω ) - {Z}_{p} ( jω )}{{Z}_{a} ( jω )} \cdot 100 \% & for & {Z}_{a} ( jω ) > {Z}_{p} ( jω ) \\ 0 & for & {Z}_{a} ( jω ) \leq {Z}_{p} ( jω )\end{matrix} \}$$

Or continuously as follows:

$$PD = \frac{{Z}_{a} ( jω ) - {Z}_{p} ( jω )}{{Z}_{a} ( jω )} \cdot 100 \%$$

As used with the methods described herein, a subject's central aortic pressure can be directly or indirectly monitored. A subject's central aortic pressure can be directly monitored, for example, with one or more pressure transducers introduced into different parts of the aorta (e.g., ascending aorta, aortic arch, thoracic aorta, abdominal aorta). For direct measurement, a pressure transducer can be, for example, positioned in the subject's aortic arch, ascending aorta thoracic aorta, or abdominal aorta. Other pressure meters and locations for their placement are known to those of skill in the art. A subject's central aortic pressure also can be determined from a signal proportional to, derived from, or a function of the subject's central aortic pressure. A signal proportional to, derived from, or a function of the subject's central aortic pressure can be measured, for example by one or more of central bioimpedence plethysmography, non-invasive tonometry, ultrasound, or pulse oximetry. Other signals proportional to or a function of a subject's central aortic pressure and methods for their measurement are known to those of skill in the art.

Also as used with the methods described herein, a subject's central aortic flow can be directly or indirectly monitored. A subject's central aortic flow can be directly monitored, for example, with one or more flow meters introduced into different parts of the aorta (e.g., ascending aorta, aortic arch, thoracic aorta, abdominal aorta). For direct measurement, a flow meter can be, for example, positioned in the subject's aortic arch, ascending aorta thoracic aorta, or abdominal aorta. Other flow meters and locations for their placement are known to those of skill in the art. The subject's central aortic flow also can be determined from a signal proportional to, derived from, or a function of the subject's central aortic flow. A signal proportional to, derived from, or a function of the subject's central aortic flow can be measured, for example by one or more of Doppler, ultrasound, bioimpedance, TEE, or Swan-Ganz Catheter. Other signals proportional to or a function of a subject's central aortic flow and methods for their measurement are known to those of skill in the art.

Further as used with the methods described herein, a subject's peripheral arterial pressure can be directly or indirectly monitored. A subject's peripheral arterial pressure can be directly monitored, for example, with one or more pressure transducers introduced into one or two radial, brachial, or femoral vessels. For direct measurement, a pressure transducer can be, for example, positioned in one or more of the subject's radial, brachial, or femoral vessels. Other pressure meters and locations for their placement are known to those of skill in the art. A subject's peripheral arterial pressure also can be determined from a signal proportional to, derived from, or a function of the subject's peripheral arterial pressure. A signal proportional to, derived from, or a function of the subject's peripheral arterial pressure can be measured, for example by one or more of central bioimpedence plethysmography, non-invasive tonometry, ultrasound, cuff blood pressure, or pulse oximetry. Other signals proportional to or a function of a subject's peripheral arterial pressure and methods for their measurement are known to those of skill in the art.

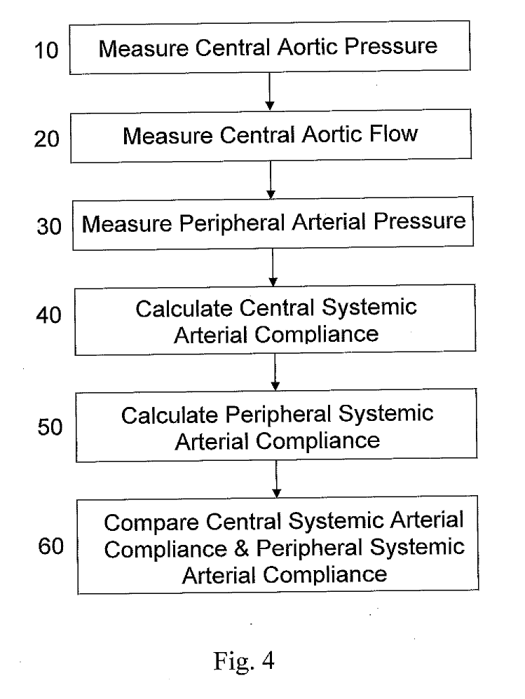

A further method for monitoring central-to-peripheral arterial pressure decoupling in a subject is shown as a flow chart in FIG. 4 and also involves measuring the central aortic pressure (10), the central aortic flow (20), and the peripheral arterial pressure (30) of a subject. Next the central systemic arterial compliance is calculated (40) using the central aortic pressure and the central aortic flow and the peripheral systemic arterial compliance is calculated (50) by using the peripheral arterial pressure and the central aortic flow. Then the central systemic arterial compliance is compared to the peripheral systemic arterial compliance (60). If the subject's peripheral systemic arterial compliance is greater than the subject's central systemic arterial compliance then central-to-peripheral arterial pressure decoupling is indicated. The degree of central-to-peripheral arterial pressure decoupling in subjects in which central-to-peripheral arterial pressure decoupling is indicated can be determined by subtracting the peripheral systemic arterial compliance from the central systemic arterial compliance. Whether a subject is experiencing central-to-peripheral arterial pressure decoupling and/or the degree of central-to-peripheral arterial pressure decoupling can be continuously monitored by continuously monitoring the central systemic arterial compliance and peripheral systemic arterial compliance.

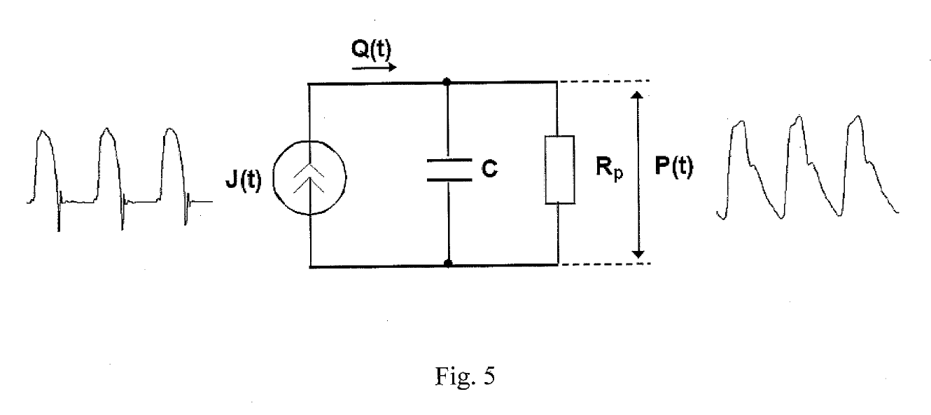

Central systemic arterial compliance can be calculated by first measuring the peripheral resistance for f=0 of the spectrum as:

$${R}_{p} = {Z}_{a} ( j0 ) = \frac{{P}_{a} ( j0 )}{{Q}_{a} ( j0 )}$$

Where Rₚ is peripheral resistance, Zₐ is systemic arterial vascular impedance, Pₐ is the power spectrum of the aortic pressure, and Qₐ is the power spectrum of the aortic flow. If we assume a two-element Compliance-Resistance model of the arterial system as shown in FIG. 5, the reactive component of the central systemic vascular impedance of the system for the first ten harmonics will be:

$${X}_{a} ( ω ) = \frac{{R}_{p} \cdot {Z}_{a} ( ω )}{{R}_{p} - {Z}_{a} ( ω )} -> [ {ω}_{1} , {ω}_{2} , … , {ω}_{10} ]$$

where the frequency, ω, is 2πf. Then the central systemic arterial compliance for the first ten harmonics is:

$${C}_{a} = \frac{1}{ω \cdot {X}_{a}} ; -> [ {ω}_{1} , {ω}_{2} , … , {ω}_{10} ]$$

(Those of skill in the art will understand that any number of harmonics can be used to measure the central systemic arterial compliance.) Similarly, the reactive component of the peripheral systemic vascular pressure impedance is:

$${X}_{p} ( ω ) = \frac{{R}_{p} \cdot {Z}_{p} ( ω )}{{R}_{p} - {Z}_{p} ( ω )} -> [ {ω}_{1} , {ω}_{2} , … , {ω}_{10} ]$$

And the peripheral systemic arterial compliance for the first ten harmonics is:

$${C}_{p} = \frac{1}{ω \cdot {X}_{p}} ; -> [ {ω}_{1} , {ω}_{2} , … , {ω}_{10} ]$$

Whether a subject's peripheral systemic arterial compliance is greater than the subject's central systemic arterial compliance (i.e., whether central-to-peripheral arterial pressure decoupling is indicated) can be expressed mathematically as follows:

PDI=Cₚ>Cₐ

The degree of central-to-peripheral arterial pressure decoupling in subjects in which central-to-peripheral arterial pressure decoupling is indicated can be expressed mathematically as follows:

$$PD = \{ \begin{matrix}{C}_{p} - {C}_{a} & for & {C}_{p} > {C}_{a} \\ 0 & for & {C}_{p} \leq {C}_{a}\end{matrix} \}$$

In this relationship the degree of the peripheral decoupling will be shown as 0 when the peripheral systemic arterial compliance is lower than the central systemic arterial compliance (i.e. no peripheral decoupling is indicated) and the degree of peripheral decoupling will be a equal to the difference between the peripheral systemic arterial compliance and the central systemic arterial compliance when the peripheral systemic arterial compliance is greater than the central systemic arterial compliance (i.e. peripheral decoupling is indicated).

Further, the difference between the peripheral systemic arterial compliance and the central systemic arterial compliance can be measured continuously, which will indicate the degree of the difference the difference between the peripheral systemic arterial compliance and the central systemic arterial compliance:

PD=Cₚ−Cₐ

When using this relationship, if PD is greater than zero, peripheral pressure decoupling is indicated, and the greater the value of PD, the greater the peripheral decoupling. When PD is less than zero, normal conditions are indicated. If PD is less than zero by more than 25%, peripheral vasoconstriction will be indicated.

The degree of peripheral decoupling could be given in % as follows:

$$PD = \{ \begin{matrix}\frac{{C}_{p} - {C}_{a}}{{C}_{a}} \cdot 100 \% & for & {C}_{p} > {C}_{a} ( jω ) \\ 0 & for & {C}_{p} \leq {C}_{a}\end{matrix} \}$$

Or continuously as follows:

$$PD = \frac{{C}_{p} - {C}_{a}}{{C}_{a}} \cdot 100 \%$$

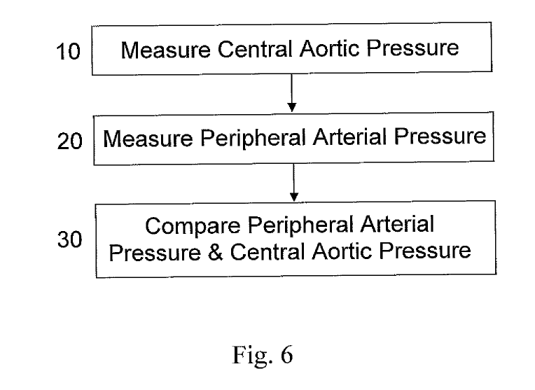

An additional method for monitoring central-to-peripheral arterial pressure decoupling in a subject is shown as a flow chart in FIG. 6 and also involves measuring the central aortic pressure (10) and the peripheral arterial pressure (20) of a subject. Next the central aortic pressure is compared to the peripheral arterial pressure (30). If the subject's peripheral arterial pressure is less than the subject's central aortic pressure then central-to-peripheral arterial pressure decoupling is indicated. The degree of central-to-peripheral arterial pressure decoupling in subjects in which central-to-peripheral arterial pressure decoupling is indicated can be determined by subtracting the central aortic pressure from the peripheral arterial pressure. Whether a subject is experiencing central-to-peripheral arterial pressure decoupling and/or the degree of central-to-peripheral arterial pressure decoupling can be continuously monitored by continuously monitoring the central aortic pressure and peripheral arterial pressure.

Whether a subject's central aortic pressure is greater than the subject's peripheral arterial pressure (i.e., whether central-to-peripheral arterial pressure decoupling is indicated) can be expressed mathematically as follows:

PDI=Pₐ(t)>Pₚ(t)

The degree of central-to-peripheral arterial pressure decoupling in subjects in which central-to-peripheral arterial pressure decoupling is indicated can be expressed mathematically as follows:

$$PD = \{ \begin{matrix}{P}_{a} ( t ) - {P}_{p} ( t ) & for & {P}_{a} ( t ) > {P}_{p} ( t ) \\ 0 & for & {P}_{a} ( t ) \leq {P}_{p} ( t )\end{matrix} \}$$

As in the methods described above, the degree of peripheral decoupling could be measured continuously as the difference between the central aortic pressure and the peripheral arterial pressure:

PD=Pₐ(t)−Pₚ(t)

When using this relationship, if PD is greater than zero, peripheral pressure decoupling is indicated, and the greater the value of PD, the greater the peripheral decoupling. When PD is less than zero, normal conditions are indicated. If PD is less than zero by more than 25%, peripheral vasoconstriction will be indicated.
The degree of peripheral decoupling could be indicated in % as follows:

$$PD = \{ \begin{matrix}\frac{{P}_{a} ( t ) - {P}_{p} ( t )}{{P}_{a} ( t )} \cdot 100 \% & for & {P}_{a} ( t ) > {P}_{p} ( t ) \\ 0 & for & {P}_{a} ( t ) \leq {P}_{p} ( t )\end{matrix} \}$$

Or continuously:

$$PD = \frac{{P}_{s} ( t ) - {P}_{p} ( t )}{{P}_{a} ( t )} \cdot 100 \%$$

The difference between a subject's peripheral arterial impedance, compliance, or pressure, and the subject's central aortic impedance, compliance, or pressure can be continually monitored. Additionally, the degree of central-to-peripheral arterial pressure decoupling can be monitored, once decoupling is indicated, by calculating the difference between the subject's peripheral arterial impedance, compliance, or pressure, and the subject's central aortic impedance, compliance, or pressure. This difference in peripheral arterial impedance, compliance, or pressure, and central aortic impedance, compliance, or pressure also can be monitored continuously. Further, the difference between a subject's peripheral arterial impedance, compliance, or pressure, and the subject's central aortic impedance, compliance, or pressure can be displayed on a graphical user interface. For example, the difference can be displayed as a bar graph or a trend graph. When central-to-peripheral arterial pressure decoupling is detected, a user can be alerted, for example, by publishing a notice on a graphical user interface or by emitting a sound.

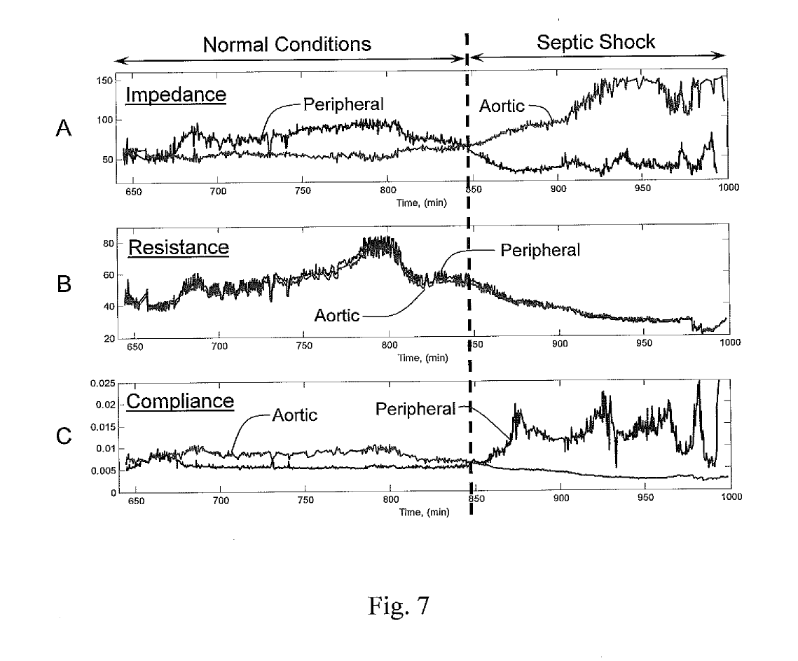

Examples of monitoring peripheral and aortic impedance and compliance (as well as resistance for a comparison) are shown in FIGS. 7A-C. FIG. 7A shows aortic impedance (Zₐ) and peripheral impedance (Zₚ) for a subject experiencing normal conditions and then experiencing central-to-peripheral arterial pressure decoupling. Similarly, FIG. 7C shows aortic compliance (Cₐ) and peripheral compliance (Cₚ) for a subject experiencing normal conditions and then experiencing central-to-peripheral arterial pressure decoupling. For comparison purposes, FIG. 7B shows aortic and peripheral resistance for the same subject (the resistance measurements do not provide a distinct indication of central-to-peripheral arterial pressure decoupling).

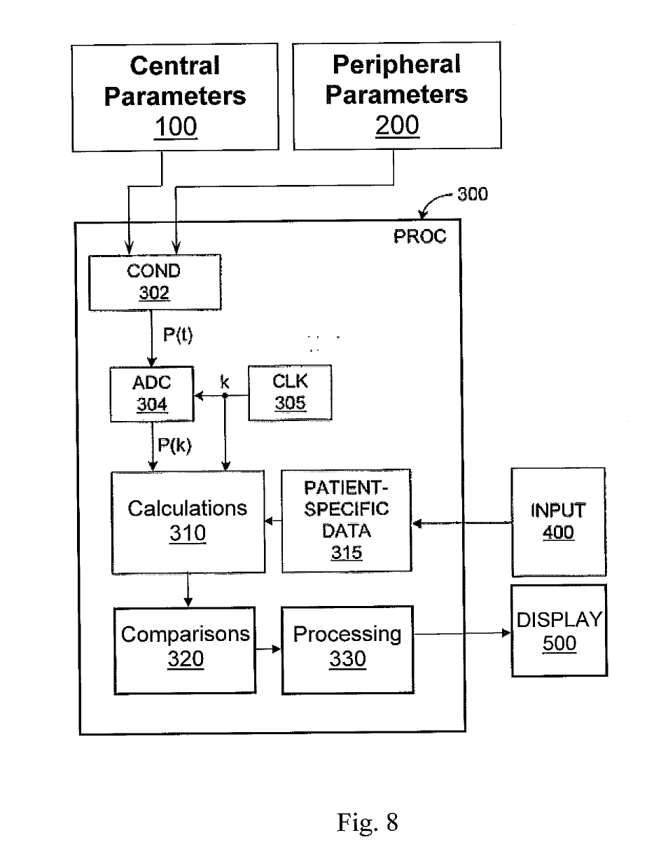

FIG. 8 shows the main components of a system that implements the methods described herein for monitoring central-to-peripheral decoupling in a subject. The methods may be implemented within an existing patient-monitoring device, or it may be implemented as a dedicated monitor. As is mentioned above, peripheral arterial pressure and/or flow (or some other input signal proportional to peripheral arterial pressure and/or flow), and central aortic pressure and/or flow (or some other signal proportional to central aortic pressure and/or flow), may be sensed in either or, indeed, both, of two ways: invasively and non-invasively. For simplicity, the system is described as having inputs for central parameters and peripheral parameters.

FIG. 8 shows central parameters (e.g., pressure and flow data) being input from box 100 and peripheral parameters being input from box 200. The central parameter 100 and peripheral parameter 200 inputs are passed via any known connectors to a processing system 300, which includes one or more processors and other supporting hardware and system software (not shown) usually included to process signals and execute code. The methods described herein may be implemented using a modified, standard, personal computer, or may be incorporated into a larger, specialized monitoring system. For use with the methods described herein, the processing system 300 also may include, or is connected to, conditioning circuitry 302 which performs normal signal processing tasks such as amplification, filtering, or ranging, as needed. The conditioned, sensed input pressure signal P(t) is then converted to digital form by a conventional analog-to-digital converter ADC 304, which has or takes its time reference from a clock circuit 305. As is well understood, the sampling frequency of the ADC 304 should be chosen with regard to the Nyquist criterion so as to avoid aliasing of the pressure signal (this procedure is very well known in the art of digital signal processing). The output from the ADC 304 will be the discrete pressure signal P(k), whose values may be stored in conventional memory circuitry (not shown).

The values P(k) are passed to or accessed from memory by a software module 310 comprising computer-executable code for implementing one or more aspects of the methods as described herein. The design of such a software module 310 will be straight forward to one of skill in the art of computer programming. Additional comparisons and/or processing as used by a method can be performed in additional modules such as 320 and 330.

If used, signal-specific data such as a record of difference values or other calculations can be stored in a memory region 315, which may also store other data or parameters as needed. These values may be entered using any known input device 400 in the conventional manner.

As illustrated by FIG. 8, the results may be ultimately displayed on a conventional display or recording device 500 for presentation to and interpretation by a user. As with the input device 400, the display 500 will typically be the same as is used by the processing system for other purposes.

Exemplary embodiments of the present invention have been described above with reference to block diagrams and flowchart illustrations of methods, apparatuses, and computer program products. One of skill will understand that each block of the block diagrams and flowchart illustrations, and combinations of blocks in the block diagrams and flowchart illustrations, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

The methods described herein further relate to computer program instructions that may be stored in a computer-readable memory that can direct a computer or other programmable data processing apparatus, such as in a processor or processing system (shown as 300 in FIG. 8), to function in a particular manner, such that the instructions stored in the computer-readable memory produce an article of manufacture including computer-readable instructions for implementing the function specified in the blocks illustrated in FIG. 8. The computer program instructions may also be loaded onto a computer, the processing system 300, or other programmable data processing apparatus to cause a series of operational steps to be performed on the computer, the processing system 300, or other programmable apparatus to produce a computer-implemented process such that the instructions that execute on the computer or other programmable apparatus provide steps for implementing the functions specified in the blocks. Moreover, various software modules 310, 320, and 330 can be used to perform the various calculations and perform related method steps described herein also can be stored as computer-executable instructions on a computer-readable medium in order to allow the methods to be loaded into and executed by different processing systems.

Accordingly, blocks of the block diagrams and flowchart illustrations support combinations of means for performing the specified functions, combinations of steps for performing the specified functions, and program instruction means for performing the specified functions. One of skill will understand that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

The present invention is not limited in scope by the embodiments disclosed herein which are intended as illustrations of a few aspects of the invention and any embodiments which are functionally equivalent are within the scope of this invention. Various modifications of the methods in addition to those shown and described herein will become apparent to those skilled in the art and are intended to fall within the scope of the appended claims. Further, while only certain representative combinations of the method steps disclosed herein are specifically discussed in the embodiments above, other combinations of the method steps will become apparent to those skilled in the art and also are intended to fall within the scope of the appended claims. Thus a combination of steps may be explicitly mentioned herein; however, other combinations of steps are included, even though not explicitly stated. The term “comprising” and variations thereof as used herein is used synonymously with the term “including” and variations thereof and are open, non-limiting terms.

1. A method for monitoring central-to-peripheral arterial pressure decoupling in a subject comprising:
measuring a central aortic pressure in the subject;
measuring a central aortic flow in the subject;
measuring a peripheral arterial pressure in the subject;
calculating a central systemic vascular impedance from the central aortic pressure and the central aortic flow;
calculating a peripheral systemic vascular impedance from the peripheral arterial pressure and the central aortic flow; and
comparing the central systemic vascular impendence to the peripheral systemic vascular impedance,
wherein central-to-peripheral arterial pressure decoupling is indicated if the subject's peripheral systemic vascular impedance is greater than the subject's central systemic vascular impedance.

2. A method for monitoring central-to-peripheral arterial pressure decoupling in a subject comprising:
measuring a central aortic pressure in the subject;
measuring a central aortic flow in the subject;
measuring a peripheral arterial pressure in the subject;
calculating a central systemic arterial compliance from the central aortic pressure and the central aortic flow;
calculating a peripheral systemic arterial compliance from the peripheral arterial pressure and the central aortic flow; and
comparing the central systemic arterial compliance to the peripheral systemic arterial compliance,
wherein central-to-peripheral arterial pressure decoupling is indicated if the subject's peripheral systemic arterial compliance is greater than the subject's central systemic arterial compliance.

3. A method for monitoring central-to-peripheral arterial pressure decoupling in a subject comprising:
measuring a central aortic pressure in the subject;
measuring a peripheral arterial pressure in the subject; and
comparing the subject's central aortic pressure to the subject's peripheral arterial pressure,
wherein central-to-peripheral arterial pressure decoupling is indicated if the subject's central aortic pressure is greater than the subject's peripheral arterial pressure.

4. The method of claim 1, wherein the subject's central systemic vascular impedance is calculated by calculating a power spectrum of the central aortic pressure in the subject, calculating a power spectrum of the central aortic flow in the subject, then dividing the power spectrum of the central aortic pressure by the power spectrum of the central aortic flow.

5. The method of claim 1, wherein the subject's peripheral systemic vascular impedance is calculated by calculating a power spectrum of the peripheral arterial pressure in the subject, calculating a power spectrum of the central aortic flow in the subject, then dividing the power spectrum of the peripheral arterial pressure by the power spectrum of the central aortic flow.

6.-10. (canceled)

11. The method of claim 1, wherein the subject's central aortic pressure and peripheral arterial pressure are determined from signals proportional to or a function of the subject's central aortic pressure and peripheral arterial pressure.

12. (canceled)

13. The method of claim 1, wherein the subject's central aortic flow is directly measured.

14. (canceled)

15. The method of claim 1, wherein the subject's central aortic flow is determined from a signal proportional to or a function of the subject's central aortic flow.

16.-21. (canceled)

22. The method of claim 1, wherein the difference between the subject's peripheral arterial impedance and the subject's central aortic impedance is displayed on a graphical user interface.

23. (canceled)

24. The method of claim 2, wherein the subject's central systemic arterial compliance is calculated by
calculating a power spectrum of the central aortic pressure in the subject;
calculating a power spectrum of the central aortic flow in the subject;
calculating a central systemic vascular impedance by dividing the power spectrum of the central aortic pressure by the power spectrum of the central aortic flow;
calculating the peripheral arterial resistance by dividing the pressure harmonic of the central aortic pressure at a frequency of zero by the flow harmonic of the central aortic flow at a frequency of zero;
calculating a first reactive component by multiplying the central systemic vascular impedance by the peripheral arterial resistance and dividing the result by the difference between the central systemic vascular impedance and the peripheral arterial resistance; and
then multiplying the first reactive component by the angular frequency and calculating the reciprocal of the result.

25. The method of claim 2, wherein the subject's peripheral systemic arterial compliance is calculated by
calculating a power spectrum of the peripheral arterial pressure in the subject;
calculating a power spectrum of the central aortic flow in the subject;
calculating a peripheral systemic vascular impedance by dividing the power spectrum of the peripheral arterial pressure by the power spectrum of the central aortic flow;
calculating the peripheral arterial resistance by dividing the pressure harmonic of the peripheral arterial pressure at a frequency of zero by the flow harmonic of the central aortic flow at a frequency of zero;
calculating a second reactive component by multiplying the peripheral systemic vascular impedance by the peripheral arterial resistance and dividing the result by the difference between the peripheral systemic vascular impedance and the peripheral arterial resistance; and
then multiplying the second reactive component by the angular frequency and calculating the reciprocal of the result.

26.-29. (canceled)

30. The method of claim 2, wherein the difference between the subject's peripheral systemic arterial compliance and the subject's central systemic arterial compliance is displayed on a graphical user interface.

31.-35. (canceled)

36. The method of claim 3, wherein the difference between the subject's peripheral arterial pressure and the subject's central aortic pressure is displayed on a graphical user interface.

37. (canceled)

38. The method of claim 1, further comprising alerting a user when central-to-peripheral arterial pressure decoupling is indicated.

39.-40. (canceled)

41. The method of claim 2, wherein the subject's central aortic pressure and peripheral arterial pressure are determined from signals proportional to or a function of the subject's central aortic pressure and peripheral arterial pressure.

42. The method of claim 3, wherein the subject's central aortic pressure and peripheral arterial pressure are determined from signals proportional to or a function of the subject's central aortic pressure and peripheral arterial pressure.

43. The method of claim 2, wherein the subject's central aortic flow is directly measured.

44. The method of claim 2, wherein the subject's central aortic flow is determined from a signal proportional to or a function of the subject's central aortic flow.

45. The method of claim 2, further comprising alerting a user when central-to-peripheral arterial pressure decoupling is indicated.

46. The method of claim 3, further comprising alerting a user when central-to-peripheral arterial pressure decoupling is indicated.
